(19) United States
(12) Patent Application Publication
(10) Pub. No.: US 2026/0015307 A1
(43) Pub. Date: Jan. 15, 2026

(54) IMPROVED 1,3-BUTYLENE GLYCOL PROCESS

(71) Applicant: OXEA Bishop LLC, Houston, TX (US)

(72) Inventors: Alexander Hensel, Essen (DE); Christopher Bischoff, Bay City, TX (US); Julian Oeljeklaus, Essen (DE); Srinivasan Ganapathy, Bay City, TX (US); Alessa Hinzman, Duisburg (DE); Kyle Gross, Alvin, TX (US); Briana Tankersley, Bay City, TX (US); Kevin Hunt, Corpus Christi, TX (US); Srini Kadambettu, Missouri City, TX (US); Diana Schlafer, Corpus Christi, TX (US)

(21) Appl. No.: 19/121,603

(22) PCT Filed: Sep. 26, 2023
(86) PCT No.: PCT/US2023/033715

§ 371 Date: Apr. 16, 2025

Related U.S. Application Data
(60) Provisional application No. 63/416,815, filed on Oct. 17, 2022.

(51) Int. Cl. C07C 29/156; C07C 29/152; C07C 29/154; C07C 29/80

(52) U.S. Cl. CPC C07C 29/156; C07C 29/152; C07C 29/154; C07C 29/80

(57) ABSTRACT

Disclosed is an improved method of making low-impurity 1,3-butylene glycol includes: (a) aldolizing acetaldehyde in a reactor to produce acetaldol; (b) hydrogenating the acetaldol in the presence of a hydrogenation/dehydrogenation catalyst in a hydrogenating reactor to produce a crude 1,3-butylene glycol stream with an active hydrogenation/dehydrogenation catalyst content; (c) removing or deactivating catalyst in the crude 1,3-butylene glycol stream; and (d) distilling the treated crude 1,3-butylene glycol stream in a distillation train to provide a purified 1,3-butylene glycol product.

### CLAIM FOR PRIORITY

This application is a National Phase Application of Application no. PCT/US2023/033715, filed 26 Sep. 2023, which was based on U.S. Provisional Application No. 63/416,815, filed 17 Oct. 2022. The priorities of the foregoing applications are hereby claimed and their disclosures incorporated herein by reference.

### TECHNICAL FIELD

The present invention relates to the manufacture of 1,3-butylene glycol and especially to high purity products suitable for cosmetic applications or applications where low-odor material is desired.

### BACKGROUND

1,3-butylene glycol is used in the manufacturing of plasticizers, polyol esters, and cosmetics. The product is conventionally produced through the aldolization of acetaldehyde, hydrogenation of the aldol in the presence of a hydrogenation/dehydrogenation catalyst, followed by purification of the crude product. Low odor, high purity product is required for cosmetic applications and is desirable in many other end-uses.

US 2003/0018224 (Tsuji et al.) discloses various processes for making high purity 1,3-butylene glycol through hydrogenation of acetaldols in the presence of a Raney Nickel catalyst and distilling the crude mixture. It is mentioned that relatively high activity Raney Nickel catalyst achieves the desired results.

U.S. Pat. No. 6,376,725 to Tsuji et al. discloses a process for making high purity 1,3-butylene glycol having low amounts of by-products and low odor. The process includes adding a base to crude 1,3-butylene glycol free of high boiling material, heat-treating the mixture and then distilling off 1,3-butylene glycol; and distilling off low-boiling materials from 1,3-butylene glycol.

GB 1205689 (Celanese) discloses a process for making low-odor 1,3-butylene glycol, including distilling the product with vessels made of stainless steel to avoid catalytic degradation of the product by iron oxides and the like.

US 2005/0154239 (Windhorst et al.) discloses a method of making 1,3-butylene glycol with low impurities by using acetaldehyde feed which is low in acid content. The process is reported to have relatively higher yields obtained by minimizing by-products.

U.S. Pat. No. 8,445,733 to Windhorst et al. discloses a method of reducing odor of 1,3-butylene glycol by treating the product with activated carbon.

U.S. Pat. No. 5,345,004 to Nishiguchi discloses a process for making cosmetic grade 1,3-butylene glycol having low amounts of by-products and low odor. The process removes croton aldehyde from the recirculated acetaldehyde.

### SUMMARY OF INVENTION

The present invention provides an improved process for the production of 1,3-butylene glycol, sometimes referred to herein as 1,3 BG, by eliminating the source of impurities in order to make cosmetic grade, low odor 1,3 BG.

In connection with the present invention, it was found that very low levels of hydrogenation/dehydrogenation catalyst residue, for instance, Raney Nickel greater than 100 ppm, in a crude product stream catalyzes the Guerbet reaction during purification of 1,3 BG and adversely impacts product quality. Conventional catalyst removal procedures like settling and filtration with conventionally employed filtration systems do not achieve the necessary reduction of the previously used catalyst in the crude product. Without intending to be bound by theory, it is evident that catalytic generation of key by-products occurs by way of the Guerbet reaction as is shown in Scheme 1 below, Scheme 2 hereinafter and the examples which follow.

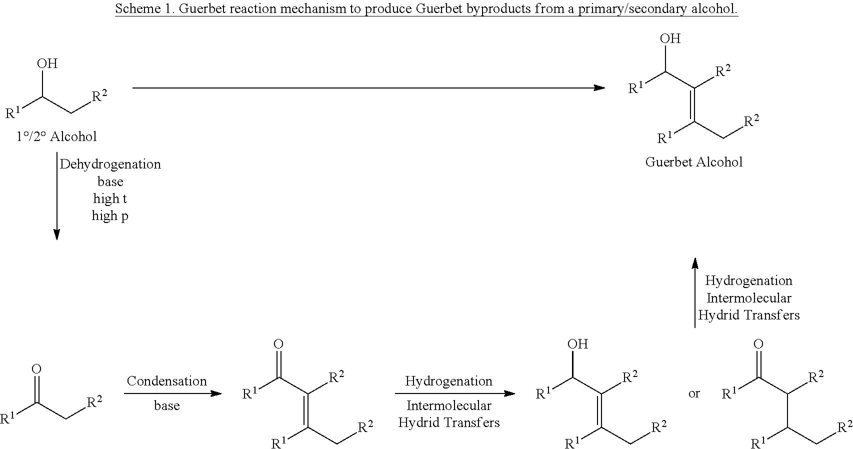

It is seen in Scheme 1, Scheme 2, and the following examples that 1,3 BG decomposes with the release of hydrogen in the presence of water and nickel at elevated temperatures. Large amounts of 2-propanol, 2-butanol, 1-butanol, as well as significant amounts of 4-hydroxy-2-butanone and methyl vinyl ketone, are detected. These molecules are key intermediates in the various by-product formation routes that have been identified by the inventors herein (including Pyrans and other ethers). Without being limited to the shown structures, some representative impurities observed include the following:

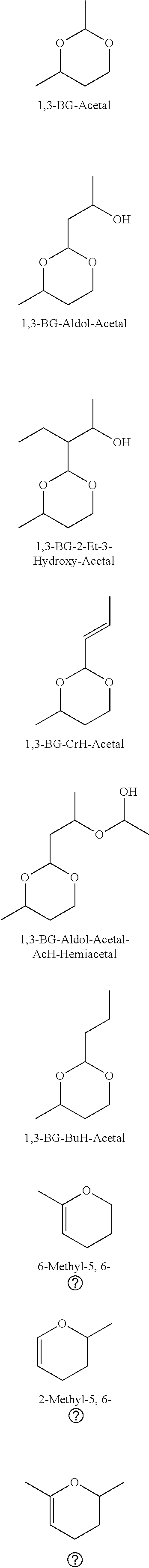

The conditions applied in the examples herein are equal and comparable to the ones applied in the purification section of a typical commercial unit.

Active hydrogenation/dehydrogenation catalyst in the crude product, at low levels, is the reason for this decomposition (no reaction in the absence of catalyst). The Guerbet reaction was first recognized in 1899 and associated with diols in the 1960's. See M. Guerbet: Action de l′alcool amylique de fermentation sur son dérivé sodé. In: Comptes rendus de l′Académie des sciences. Band 128, 1899, S. 511-513. In the Examples, it is seen the reaction is initiated and catalyzed by Ni. For general information on Guerbet reactions see GB 761296 (Esso Research and Engineering Co.), page 2, Col. 2, line 92 to page 3, Col. 1, line 32.

There is provided in one aspect of the present invention, a method of making 1,3-butylene glycol comprising:
(a) aldolizing acetaldehyde in a reactor to produce acetaldol;
(b) hydrogenating the acetaldol in the presence of a hydrogenation/dehydrogenation catalyst in a hydrogenating reactor to produce a crude 1,3-butylene glycol stream with an active hydrogenation/dehydrogenation catalyst content of greater than 100 ppm;
(c) removing or deactivating catalyst in the crude 1,3-butylene glycol stream to provide a treated crude 1,3-butylene glycol stream with less than 100 ppm active hydrogenation/dehydrogenation catalyst; and
(d) distilling the treated crude 1,3-butylene glycol stream in a distillation train to provide a purified 1,3-butylene glycol product.

In another aspect of the invention there is provided an improvement to a continuous process for making 1,3-butylene glycol of the class including hydrogenating acetaldol in the presence of a hydrogenation/dehydrogenation catalyst in a hydrogenating reactor to produce a crude 1,3-butylene glycol stream with an active hydrogenation/dehydrogenation catalyst content and distilling the crude 1,3-butylene glycol stream in a distillation train to provide a purified 1,3-butylene glycol product, where the improvement comprises removing or deactivating active catalyst in the crude 1,3-butylene glycol stream to provide a treated crude 1,3-butylene glycol stream with less active hydrogenation/dehydrogenation catalyst than the crude 1,3-butylene glycol stream prior to treatment and within the range of from 0 to 750 ppm prior to distillation and distilling the treated crude 1,3-butylene glycol stream in a distillation train to provide the purified 1,3-butylene glycol product.

Further aspects and details are provided in the discussion which follows.

### BRIEF DESCRIPTION OF DRAWINGS

FIG. 1 illustrates a schematic flow diagram of a typical continuous production process for 1,3-butylene glycol of the invention.

### DETAILED DESCRIPTION

The invention is described in detail herein connection with the Figure for purposes of illustration, only. The invention is defined in the appended claims. Unless otherwise indicated, terminology and symbols used herein is given its ordinary meaning; for example, %, ppm and like terminology means weight percent, parts per million by weight and so forth unless otherwise indicated.

“Consisting essentially of” and like terminology refers to the recited components and excludes other ingredients which would substantially change the basic and novel characteristics of the composition, article, or process. Unless otherwise indicated or readily apparent, a composition or article consists essentially of the recited or listed components when the composition or article includes 90% or more by weight of the recited or listed components. That is, the terminology excludes more than 10% unrecited components. Any of the products disclosed and claimed herein may consist essentially of the recited components.

“Effective Pore Size” of a filter system refers to the filter system's ability to filter out particles of a certain size. For example, a 0.20 micron (μm) rated filter system will remove particles with a diameter of 0.2 microns or larger from a filtration stream.

Filter, Filter System and like terminology refer to a single filter element or multiple filter elements including filter elements arranged in series or parallel characterized by an Effective Pore Size. Such filters include without limitation, leaf-type systems, cartridge-type systems, bag-type systems, centrifugal-type systems, settling-type systems, candle-type systems, and/or magnetic-type systems. It is preferred to employ Filtration Systems having a Primary Filtration System followed by a Secondary or Polishing Filter System with a smaller Effective Pore Size. The Primary Filtration may consist of a settling device with or without one or more filter(s) of the types disclosed above. Polishing Filter Systems can include, but not limited to, those mentioned above. Additionally, pre-coat materials can be added to enhance both the primary and secondary system filtration capabilities. Pre-coat type materials can be of a variety of type including diatomaceous earth, perlite, and/or cellulose. Polishing Filter Systems preferably have an Effective Pore size of less than or equal to 1 micron.

“Guerbet Impurities” or “Guerbet Byproducts” include 2-propanol, 2-butanol, 1-butanol, 4-hydroxy-2-butanone, methyl vinyl ketone and byproducts generated with these molecules, such as pyrans and other ethers, representative byproducts including the following:

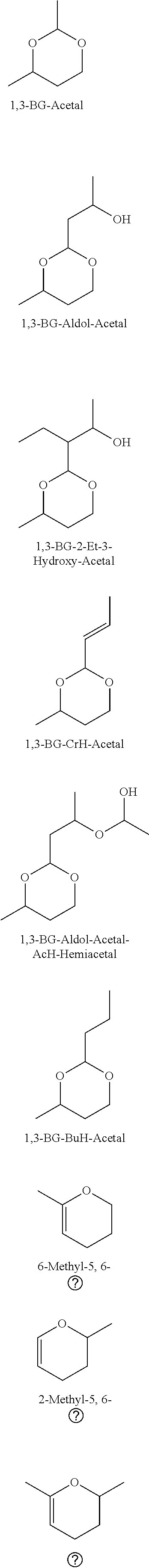

As used herein, “hydrogenation/dehydrogenation catalyst” and like terminology refers to metallic catalysts used for hydrogenating and dehydrogenating organic compounds, including transition metal catalysts selected from the list of Ti, Zr, V, Nb, Cr, Mo, Mn, Re, Fe, Ru, Os, Co, Rh, Ir, Ni, Pd, Pt, Cu, Ag, Au, Zn, Cd and Hg metals. The catalyst may be in the form of a fixed bed, optionally catalyst metal supported on a carrier, or in the form of a slurry of supported or unsupported catalyst metal. Particularly preferred are Raney catalysts in slurry form, selected from Raney-Co, Raney-Ni, Raney-Cu, Raney-Fe, which include metals that might be included in the Raney catalysts as promotor during the manufacture process of the same, notably but without being bound to, Al, Zn and Cr.

A typical, 1,3 BG process can be separated and simplified into three distinct steps. First aldolization of acetaldehyde towards 3-hydroxybutanal, second hydrogenation of the latter to the corresponding crude diol, and third purification of the crude 1,3-butylene glycol. The process can be carried out in either batch mode, semi-continuous mode or more preferably is carried out in a 100% continuous manner from the acetaldehyde feed to the final purification of the product.

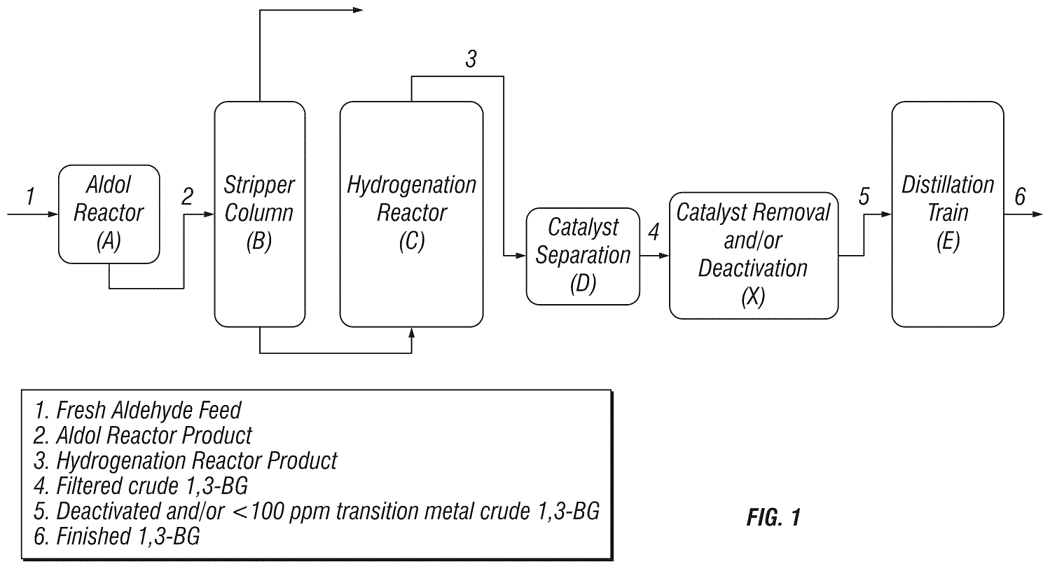

A simplified overall process flow for a continuous process of the invention is shown in FIG. 1 which shows schematically an apparatus for producing 1,3-butylene glycol having reactors, purification towers and removal units described below.

### Aldolization of Acetaldehyde:

In describing the process, reference is made to the simplified process flow in FIG. 1. The unit feeds acetaldehyde to an aldolization reactor A. 2-20%, more suitably 2-10% caustic is added to the reactor. The aldolization reactor A operates at conversions, between 10-90%, more preferably between 20-80% or even more preferably between 22-62%. Conversion is controlled through the typical process parameters, known to the ones skilled in the art. Temperature is controlled between-1° C.-54° C. (30-130° F.), more preferably between 10° C.-38° C. (50-100° F.), even more preferably between 16° C.-32° C. (60-90° F.) such as between 21° C. (70° F.) and 29° C. (85° F.) with a reaction pressure of ranging from 138 kPa-483 kPa (20-70 psig), more suitably from 207 kPa-414 kPa (30-60 psig), even more suitably from 172 kPa-345 kPa (25-50 psig).

Reactor product is withdrawn and sent to a stripper column to remove light ends from the product stream. Stripper tower B residue contains the intermediate that is fed forward into the hydrogenation section of the unit.

### Hydrogenation of the Acetaldol:

Hydrogenation is accomplished using a metal based catalyst, more preferably from the list of Ti, Zr, V, Nb, Cr, Mo, Mn, Re, Fe, Ru, Os, Co, Rh, Ir, Ni, Pd, Pt, Cu, Ag, Au, Zn, Cd and Hg metals, even more preferably from the list of Raney-Co, Raney-Ni, Raney-Cu, Raney-Fe or similar catalysts. The hydrogenation reactor C operates typically at 66° C.-121° C. (150-250° F.), more suitably at 82° C.-110° C. (180-230° F.), even more suitably at 88° C.-104° C. (190-220° F.) and 3447 kPa-5516 kPa (500-800 psig), more preferably 4137 kPa-5171 kPa (600-750 psig), even more preferably 4482 kPa-4964 kPa (650-720 psig). Hydrogen is fed to the reactor and can be carried through the reactor as for example a two-phase flow with the intermediate and active hydrogenation catalyst. Hydrogenation product and some active hydrogenation catalyst come out of the reactor. Unreacted hydrogen is separated from the crude reaction mixture, while the liquid phase is sent to a catalyst separation system D. The active catalyst is removed, conventionally by decanting and optionally by filtration. The crude reaction product moves forward to purification E.

### Purification (E):

The pretreated crude 1,3 BG coming out of hydrogenation contains both light and heavy impurities. The crude usually contains already high amounts of 1,3-butylene glycol in water together with some light ends like ethanol and/or butanol and/or crotonaldehyde and corresponding impurities, Heavy end impurities include 2,6-dimethyl-1,3-dioxan-4-ol (Aldoxane), 2-ethyl-1,3-butylene glycol and/or 2,4-dimethyl-1,3-dioxane (BG Acetal) and/or 3-hydroxybutyl acetate (BG Monoacetate). Water can make up 50-90% of the crude product stream, suitably 60-80% and even more suitably 65-75% with the remainder consisting essentially of 1,3-butylene glycol.

The impurities can be removed in purification through a series of towers. In one variation of the invention, the first purification step might include removal of any light end impurities including water. In another variation of the invention, second purification step might include removal of any heavy ends followed by a third and/finishing step to provide high quality 1,3-butylene glycol without odor. By means of this invention, the corresponding purification steps might include the use of vacuum flashers.

Catalyst Separation Unit in Accordance with the Invention (X, FIG. 1):

However, in accordance with the invention, an additional or substituting the aforementioned catalyst separation train catalyst removal/deactivation, a unit (X), is provided to reduce the active hydrogenation/dehydrogenation catalyst level to less than 1000 ppm, more preferably less than 500 ppm, even more preferably less than 150 ppm, 100 ppm or less prior to forwarding a treated crude 1,3-butylene glycol stream to further purification.

For the purpose of pre-treating the crude reaction product in order to decrease the level of active hydrogenation/dehydrogenation catalyst, it is advised as part of the invention to utilize a primary and secondary catalyst deactivation and/or removal system.

Primary catalyst deactivation and/or removal system can include a filtration system, but not limited to, leaf-type systems, cartridge-type systems, bag-type systems, centrifugal-type systems, settling-type systems, candle-type systems, and/or magnetic-type systems with or without a settling device. Secondary catalyst deactivation system or Polishing Filter Systems can include, but not limited to, those mentioned prior. Additionally, pre-coat materials can be added to enhance both the Primary and Polishing Filter System filtration capabilities. Pre-coat type materials can be of a variety of type including diatomaceous earth, perlite, and/or cellulose.

Deactivating the transition metal based hydrogenation/dehydrogenation catalyst is another viable route to prevent decomposition of the desired 1,3-butylene glycol and formation of unwanted byproducts by means of the Guerbet reaction. Without being bound to theory it is believed that catalytic activity of said hydrogenation/dehydrogenation catalysts is defined by the active surface sites of the heterogeneous and/or homogeneous material, which serves as the catalyst. Thus, deactivation of the active sites could lead to suppression of unwanted side-reactions and are a central part of this invention.

Deactivating can take place by means of contacting said catalyst with hypochlorites, nitrate or nitrite based solutions, solubilized carbon monoxide, phosphines, and/or any other component that would potentially block active surface from chemisorption and/or physisorption mechanisms. Contacting crude process streams with deactivating ions prevents further byproduct formation. Crude process streams and/or process equipment can be treated continuously or non-continuously to deactivate transition metal catalyst that is present in unintended and/or intended locations.

In accordance with the invention, a finishing step may be omitted since odor-causing impurities are reduced.

### Experimental Examples

### 1. Formation of by Products:

A series of trials were performed in order to determine the effects of remaining active hydrogenation/dehydrogenation catalyst on impurity generation from 1,3-butylene glycol during the downstream processing of crude 1,3-butylene glycol.

### General Procedure

Under an inert gas atmosphere (Ar) 1,3-butylene glycol was dissolved in water (30 wt %) to represent a typical crude 1,3-butylene glycol stream, and fed to a laboratory distillation apparatus equipped with a cooling trap, a condensate collector and a gas collector. The distillation temperature was maintained at 103° C. The unit was operated with varying amounts of hydrogenation/dehydrogenation catalyst in the aqueous 1,3-butylene glycol, including a benchmark with no catalyst at all, in order to study the effect of the latter.

The gas and condensate were collected and analyzed.

TABLE 1.1
Influence of concentration of hydrogenation/dehydrogenation
catalyst on gas formation from 1,3-butylene glycol as
indication for decomposing Guerbet activity.

| | Catalyst | H₂ | O₂ | N₂ |
|---|---|---|---|---|
| Entry | [wt %] | [GC %]^{[a]} | [GC %]^{[a]} | [GC %]^{[a]} |
| 1 | — | 0 | 30.06 | 69.94 |
| 2 | 1 | 96.87 | 0.86 | 2.24 |
| 3 | 0.1 | 54.93 | 13.38 | 31.69 |
| 4 | 0.01 | 3.64 | 30.30 | 66.06 |
| 5 | 0.001 | 0 | 30.22 | 69.78 |

^{[a]}As Ar was used as inert gas, it was excluded from the results by calculation.

TABLE 1.2
Influence of concentration of hydrogenation/dehydrogenation catalyst
on formation of representative by-products from 1,3-butylene
glycol as indication for decomposing Guerbet activity.

| | Catalyst | 1,3-BG | 2-PrOH | 2-BuO | 2-BuOH | 1-BuOH | 4H2B | Rest |
|---|---|---|---|---|---|---|---|---|
| Entry | [wt %] | [GC %] | [GC %] | [GC %] | [GC %] | [GC %] | [GC %] | [GC %] |
| 1 | — | 95.5 | 0.7 | n.d. | n.d. | 0.2 | n.d. | 3.5 |
| 2 | 1 | 74.58 | 10.61 | 6.25 | 0.9 | 1.54 | 3.08 | 3.04 |
| 3 | 0.1 | 92.5 | 1 | 0.7 | 0.1 | 0.4 | 0.7 | 4.62 |
| 4 | 0.01 | 95.1 | 0.2 | 0.1 | 0.1 | n.d. | 0.1 | 4.42 |
| 5 | 0.001 | 94.7 | n.d. | n.d. | 0.1 | n.d. | n.d. | 5.22 |

Impurity generation correlates with hydrogen generation, according to the following Scheme 2:

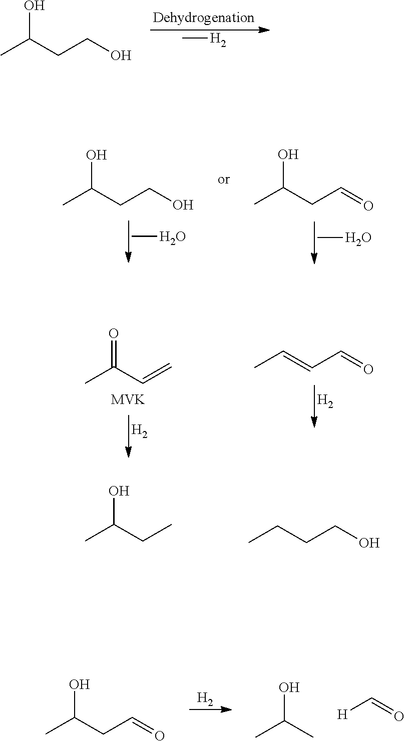

It is seen without active hydrogenation/dehydrogenation catalyst present, impurity generation is absent, but already with 1 wt. % or 0.1 wt % the generation of by-products is high enough to be detected by simple GC analysis, especially 1-butanol and 2-butanol with lesser amounts of 4-hydroxy-2-butanone (4H2B). As can be appreciated from the above scheme, 2-butanol is probably derived from 4H2B. Impurity generation abates at about 100 ppm catalyst in the mixture and is substantially absent at 10 ppm in the mixture.

Impurity generation is thus substantially improved in a commercial unit if active catalyst is either removed or deactivated to levels corresponding to less than 100 ppm active catalyst before further processing of the crude product stream, which distills the crude, treated product at elevated temperatures

### 2. Filtration to Prevent Byproducts Formation:

### Filtration Procedure:

Representative mixtures of 1,3-BG and water in the presence of 1% (w/w) transition metal catalyst were filtered using varying Effective Pore Size filtration discs. Material was passed through a single filtration disc and exposed to general procedure conditions for byproduct formation stated above. The distillate liquid was collected and analyzed.

TABLE 2.1
Influence of filtration on reducing Guerbet byproduct formation.

| | Unfiltered | 0.45 micron | 0.1 micron |
|---|---|---|---|
| 1,3-BG + | 98.00 | 99.74 | 99.97 |
| Water^{[a]} | | | |
| Guerbet | 2.00 | 0.26 | 0.03 |
| Byproducts^{[b]} | | | |

^{[a]}Reported as wt % by GC and KF-titration;
^{[b]}Reported as wt % by GC

In the foregoing examples, filter paper discs were used. One skilled in the art appreciates that Filtration Systems including polyester discs or tubes, polypropylene discs or tubes or sintered metal discs or tubes may be employed.

### 3. Deactivation to Prevent Byproduct Formation

Representative, mixtures of 1,3-BG and water were exposed to forms of deactivated and activated transition metal catalyst. Deactivation in the presence of excess deactivating ions with respect to transition metal catalyst display the difference in byproduct formation.

### Deactivation of Raney-Nickel Using NaOCl-Solution (8-10% Aqueous Solution)

Raney nickel (5 g) is added to a 250 mL round-bottom flask with water (35 g) and 8-10% aqueous NaClO-solution (˜100% excess based on Raney Nickel, 150 g) is added within 10 min. During addition, the temperature of the mixture increases from room temperature (˜20° C.) to 41° C. After complete addition the mixture is then stirred for 1 h at 60° C.
Afterwards, the mixture is cooled to room temperature and the catalyst is filtered and washed with water. The deactivated catalyst is stored wet until usage.

### Deactivation of Raney-Nickel Using NaNO₃-Solution (10% Aqueous Solution)

Raney nickel (20 g) is added to a 500 mL round-bottom flask attached with water (80 g) and of NaNO₃ (˜6% excess based on Raney Nickel, 300 g) is added within 25 min. During addition, the temperature increases from room temperature (˜20° C.) to 33° C. After complete addition the mixture is then stirred for 1 h at 60° C.

Afterwards, the mixture is cooled to room temperature and the catalyst is filtered and washed with water. The deactivated catalyst is stored wet until usage.

TABLE 3.1
Influence of deactivating hydrogenation/dehydrogenation
catalyst on gaseous formation.

| | | | | Guerbet |
|---|---|---|---|---|
| Entry | H₂ ^{[a], [b]} | O₂ ^{[a], [b]} | N₂ ^{[a], [b]} | Byproducts^{[c]} |
| Active Catalyst | 54.93 | 13.38 | 31.69 | 1.90 |
| NaOCl | 0.00 | 33.64 | 66.36 | 0.10 |
| Deactivation | | | | |
| NaNO₃ | 0.00 | 30.09 | 68.69 | 0.20 |
| Deactivation | | | | |

^{[a]} Ar used as inert;
^{[b]} Reported as gas phase wt % GC;
Liquid phase distillate wt % by GC

What is claimed is:

1. A method of making 1,3-butylene glycol comprising:
(a) aldolizing acetaldehyde in a reactor to produce acetaldol;
(b) hydrogenating the acetaldol in the presence of a hydrogenation/dehydrogenation catalyst in a hydrogenating reactor to produce a crude 1,3-butylene glycol stream with an active hydrogenation/dehydrogenation catalyst content of greater than 100 ppm;
(c) removing or deactivating catalyst in the crude 1,3-butylene glycol stream to provide a treated crude 1,3-butylene glycol stream with less than 100 ppm active hydrogenation/dehydrogenation catalyst; and
(d) distilling the treated crude 1,3-butylene glycol stream in a distillation train to provide a purified 1,3-butylene glycol product.

2. (canceled)

3. The method according to claim 1, wherein the hydrogenation/dehydrogenation catalyst comprises a transition metal hydrogenation/dehydrogenation catalyst selected from Ti, Zr, V, Nb, Cr, Mo, Mn, Re, Fe, Ru, Os, Co, Rh, Ir, Ni, Pd, Pt, Cu, Ag, Au, Zn, Cd and Hg metals.

4. The method according to claim 1, wherein the hydrogenation/dehydrogenation catalyst is a Raney catalyst selected from Raney-Co, Raney-Ni, Raney-Cu, Raney-Fe.

5. The method according to claim 1, wherein the hydrogenation/dehydrogenation catalyst is a Raney Nickel catalyst.

6. (canceled)

7. The method according to claim 1, wherein the treated crude 1,3-butylene glycol stream contains less than 75 ppm active hydrogenation/dehydrogenation catalyst.

8. (canceled)

9. (canceled)

10. (canceled)

11. The method according to claim 1, further comprising removing and/or deactivating residual catalyst from surfaces of the distillation train.

12. The method according to claim 1, wherein the active hydrogenation/dehydrogenation catalyst in the crude 1,3-butylene glycol stream is deactivated with a deactivating agent effective to block active catalytic sites on the hydrogenation/dehydrogenation catalyst.

13. (canceled)

14. The method according to claim 12 in which deactivation of said catalyst in the crude 1,3-butylene glycol stream is performed by contact of the catalyst with deactivating agents selected from hypochlorites, nitrate or nitrite based solutions, solubilized carbon monoxide and phosphines.

15. The method according to claim 1 in which the crude 1,3 butylene glycol stream containing residual active hydrogenation/dehydrogenation catalyst is filtered with a Filter System having an Effective Pore Size of from 0.01 to 1 micron.

16. The method according to claim 15, in which the Filter System is selected from the group of a leaf system, a cartridge system, a bag system, centrifugal system, settling system, a candle system, a magnetic system or combinations thereof.

17. (canceled)

18. (canceled)

19. (canceled)

20. (canceled)

21. (canceled)

22. (canceled)

23. (canceled)

24. In a continuous process for making 1,3-butylene glycol of the class including hydrogenating an acetaldol in the presence of a hydrogenation/dehydrogenation catalyst in a hydrogenating reactor to produce a crude 1,3-butylene glycol stream with an active hydrogenation/dehydrogenation catalyst content and distilling the crude 1,3-butylene glycol stream in a distillation train to provide a purified 1,3-butylene glycol product, the improvement comprising removing or deactivating active catalyst in the crude 1,3-butylene glycol stream to provide a treated crude 1,3-butylene glycol stream with less active hydrogenation/dehydrogenation catalyst than the crude 1,3-butylene glycol stream prior to treatment and within the range of from 0 to 750 ppm prior to distillation and distilling the treated crude 1,3-butylene glycol stream in a distillation train to provide the purified 1,3-butylene glycol product.

25. The improvement according to claim 24, wherein the content of active hydrogenation/dehydrogenation catalyst in the treated crude 1,3-butylene glycol stream is within the range of 0 to 500 ppm prior to distillation.

26. (canceled)

27. (canceled)

28. (canceled)

29. (canceled)

30. (canceled)

31. (canceled)

32. An apparatus for producing 1,3-butylene glycol comprising:
(a) an aldolization reactor for aldolizing acetaldehyde to acetaldol;
(b) a hydrogenation reactor coupled to the aldolization reactor containing a slurry hydrogenation/dehydrogenation catalyst for hydrogenating acetaldol from the aldolization reactor and being operative to provide a crude 1,3-butylene glycol stream containing active hydrogenation/dehydrogenation catalyst;
(c) a catalyst removal/deactivation unit coupled to the hydrogenation reactor adapted to remove or deactivate active hydrogenation/dehydrogenation catalyst in the crude 1,3-butylene glycol stream effective to remove or deactivate active catalyst in the crude 1,3-butylene glycol stream to provide a treated crude 1,3-butylene glycol stream with less active hydrogenation/dehydrogenation catalyst than the crude 1,3-butylene glycol stream prior to treatment in the catalyst removal/deactivation unit and within the range of from 0 to 750 ppm; and
(d) a distillation train coupled to the catalyst removal/deactivation unit for purifying the treated crude 1,3-butylene glycol product stream.

33. The apparatus according to claim 32, wherein the catalyst removal/deactivation unit is operative to reduce the content of active hydrogenation/dehydrogenation catalyst in the crude 1,3-butylene glycol stream so that the treated crude 1,3-butylene glycol stream has a content of active hydrogenation/dehydrogenation catalyst within the range of 0 to 500 ppm.

34. (canceled)

35. (canceled)

36. (canceled)

37. (canceled)

38. (canceled)

39. The apparatus according to claim 32 in which the crude 1,3 butylene glycol stream containing residual active hydrogenation/dehydrogenation catalyst is filtered with a Primary Filter System followed by filtering with a Polishing Filter System in the catalyst removal/deactivation unit.

40. The apparatus according to claim 39 in which the crude 1,3 butylene glycol stream containing residual active hydrogenation/dehydrogenation catalyst is filtered with a Polishing Filter System in the catalyst removal/deactivation unit having an Effective Pore Size of from 0.01 to 1 micron.

41. The apparatus according to claim 39, in which the Polishing Filter System is selected from a leaf system, a cartridge system, a bag system, centrifugal system, settling system, a candle system, a magnetic system or combinations thereof.

42. The apparatus according to claim 39, wherein the Polishing Filter System has an Effective Pore Size of from 0.01 to 0.5 microns.

43. (canceled)

44. (canceled)

45. The apparatus according to claim 32, wherein active hydrogenation/dehydrogenation catalyst in the crude 1,3-butylene glycol stream is deactivated in the catalyst removal/deactivation unit with a deactivating agent effective to block active catalytic sites on the hydrogenation/dehydrogenation catalyst.

46. The apparatus according to claim 45, wherein the active hydrogenation/dehydrogenation catalyst in the crude 1,3-butylene glycol stream is deactivated with a molar excess of the deactivating agent with respect to the catalyst present in the crude 1,3-butylene glycol stream.

47. (canceled)
